(19) United States
(12) Patent Application Publication
(10) Pub. No.: US 2004/0001888 A1
(43) Pub. Date: Jan. 1, 2004

(54) Solid dosage forms for rapid dissolution of poorly soluble drugs

(75) Inventor: Tuo Jin, Highland Park, NJ (US)

Correspondence Address: Albert Wai-Kit Chan, Law Offices of Albert Wai-Kit Chan, LLC, World Plaza, Suite 604, 141-07 20th Avenue, Whitestone, NY, 11357, US

(73) Assignee: BioPharm Solutions Inc.

(21) Appl. No.: 10/606,344

(22) Filed: Jun. 26, 2003

Related U.S. Application Data
(60) Provisional application No. 60/391,756, filed on Jun. 26, 2002.

(51) Int. Cl. A61K 9/20

(52) U.S. Cl. 424/465

(57) ABSTRACT

This invention demonstrated novel pharmaceutical compositions that improve dissolution, water dispersion and/or oral absorption of insoluble or poorly soluble drugs without increase in formulation complicity and patient appliance as compared with conventional solid-dosage form. The compositions of the present invention comprise a lipid or mixed lipids that dissolve the insoluble or poorly soluble drugs and forms solution, micelles, microemulsion or emulsion with the drugs in aqueous media. The compositions further comprise a porous powder or mixed porous powder that absorb the drug-lipid melts in a considerable amount (>than their own mass) while remaining free flowing and compressible in nature. Due to their excellent effectiveness-simplicity ratio, the compositions of this invention have a wide applicability to therapeutic compounds whose efficacy is limited by poor solubility, low dissolution rate and less absorption.

This application claims priority of U.S. Serial No. 60/391,756 filed Jun. 26, 2002, the content of which is incorporated here into this application.

Throughout this application, various references are referred to and disclosures of these publications in their entireties are hereby incorporated by reference into this application to more fully describe the state of the art to which this invention pertains.

### BACKGROUND OF THE INVENTION

Despite extensive research efforts, solubility and dissolution rate remain key problems in drug discovery and product development for oral dosage forms [1]. Compounds that have limited solubility, in water, typically below 0.1 mg/ml, present unusual challenges in drug discovery [2]. This is especially true in the many circumstances for which solubilization and dissolution limit drug absorption. Strategies for improving apparent solubility and dissolution rate include forming soluble salts for ionizable drugs [3], reducing crystal size [4], forming soluble pro-drugs, using amorphous forms [5], co-solvents and superdisintegrants [6], impregnating liquid drugs or drug solution in porous powders [7] and using surface active self-emulsifying systems [8]. Although salt formation and particle size reduction are commonly used to increase dissolution rate and oral absorption, there are practical limitations for these techniques. The salt formation is not feasible for neutral compounds and weak electrolytes. Very fine powders of hydrophobic drugs, on the other hand, are difficult to disperse in water due to the poor wettability of the particle surfaces [9]. The loading of liquid drugs in porous powder (called “powder solutions” by Sheth and Jarowski) encountered flow property and compressibility problems in pharmaceutical manufacture, so that it is only suitable for low-dose drugs [7]. Among the strategies, those that increase dissolution rate of poorly soluble drugs with lipids/surfactants are the most commonly used techniques for enhancing their absorption. Lipid-based self-emulsifying systems are particularly interesting in that they offer both kinetic (dissolution) and thermodynamic (micro-emulsification) enhancement of drug absorption [1]. By forming micelles or microemulsions with the drug substances, lipid molecules may, not only facilitate dissolution, but also increase apparent solubility.

For lipid-based dissolution enhancement, poorly soluble drugs are first dissolved in liquid lipid-melts and formulated as soft or hard capsules [8,9]. This lipid-based hard gel capsule technology is regarded a breakthrough in sense of that it overcomes the scale-up difficulties in solid dispersion as well as avoids retarded dissolution due to the loss of solubilizer and formation of a drug-rich surface layer [9]. However, when the shell of such a capsule disintegrates after oral ingestion, the drug-lipid matrix is exposed to the gastrointestinal fluid as a solid plug, and drug dissolution may be limited by surface erosion of this solid plug. It is reasonable to believe that the dissolution or solubilization may further be improved if the drug-lipid matrix is pre-dispersed into micro- or nanometer-size before final formulation. Pather et al. demonstrated that a pre-formed drug-lipid microemulsion was absorbed in porous powders to form a solid form. Self-emulsifying powders were also prepared by freeze-drying drug-lipid emulsions or microemulsions [10]. Similarly, insoluble drugs were also formed microemulsion with lipids, followed by freeze-drying as solid dosage forms [11]. However, these processes will encounter the scale-up complicity or the need of organic solvents. A lipid-based method that facilitates dissolution and, at the same time, achieves manufacturing simplicity will be fascinating.

### SUMMARY OF THE INVENTION

This invention has demonstrated novel pharmaceutical compositions that improve apparent solubility, dissolution rate and absorption of drugs which are poorly soluble. These novel compositions comprise non-aqueous solutions of drugs with self-emulsifying agents, such as Gelucira, Vitamin E TPGS or other lipid systems, that are absorbed into porous solid materials which may be further formulated into solid dosage forms.

These compositions differ from the so called “microemulsions as solid dosage forms” [10] in that the drugs to be loaded do not need to form microemulsions prior to loading into solid material, thus formulation procedures are much simpler. These compositions are also different from the Gelucira hard gel capsules [9] for which dissolution is based on a passive diffusion after surface erosion of a solid plug. This type of solid dosage forms utilizes a mechanism to “actively” squeeze a hydrated lipids-drug matrix form the porous carrier to the solution, thus the release process and emulsification process can be achieved at the same time. Our preliminary experiment showed that initial dissolution rate of a model drug was four times faster than a Gelucire hard capsule.

For the mechanistic details of these compositions, we hypothesize that the drug-lipid melt can be absorbed into the hydrophilic pores of porous materials of nanometer-sized compartments (the pores) and cooled to solid. Upon absorbing water, the size-limited lipid-drug matrix will swell and, squeeze itself out of the nanometer-sized pore structure. This mechanism is feasible for poorly soluble or insoluble drugs that form fine emulsions with lipid molecules.

These compositions preserve all the manufacturing conveniences and patient compliances of pharmaceutical solid dosage forms with comparable effectiveness to other solubility-improving approaches of insoluble drugs. To name a few, the drug loading process is much simpler and the loading capacity larger compared with the microemulsion-loaded solid powders [10] and the so called powder solutions [7]. Sufficient loading capacity of drugs into porous powders ensures that a tablet will not be too big for a given dose. These compositions (after drug loading) also possess good flow property and compressibility which is not found in lyophilized microemulsions [11]. The drug-loaded powders in the present invention showed no difference as pharmaceutical granules in the tableting process.

In addition, these compositions offer good polymorph-stability of drugs in that the drugs will not re-crystallize during the storage period, as often found for amorphous drug-lipid solid melts [9]. For the present invention, the drug-lipid matrix is isolated in each nanometer-sized pore, thus the amount of drug molecules accessible to each other to form crystals is insufficient.

The power of the present compositions in solubilizing (or apparently solublizing) poorly soluble drugs may still be limited by the same factors found in other emulsion- or microemulsion-based approaches. For example, the drugs to be loaded must be soluble in melted lipids, and the lipids themselves must be soluble or dispersible in water to form emulsion or microemulsion with the drug. However, the present compositions possess no compromise on the aspects found in other lipid-based solubilization strategies.

### BRIEF DESCRIPTION OF THE FIGURES

FIG. 1. Schematic description of drug dissolution from an active-release tablet.

FIG. 2. Dissolution profiles of triamterene formulated in the form of active-release tablet, reference tablet, drug-lipid plug and unformulated drug powder in 0.1 M HCl. -□-: active-release tablet; -▴-: reference tablet; -▪-: plug capsule; -●-: drug-only capsule.

FIG. 3. Dissolution profiles of triamterene formulated in the form of active-release tablet, reference tablet, drug-lipid plug and unformulated drug powder in water. -□-: active-release tablet; -▴-: reference tablet; -▪-: plug capsule; -●-: drug-only capsule.

FIG. 4. Dissolution profiles of triamterene formulated in the form of active-release tablet in 0.1 M HCl and in water.

A: dissolution in 0.1 HCl; B: dissolution in water.

-□-: 30 mg triamterene loaded in 350 mg Gelucire, 150 mg silica and 50 mg alumina;

-□-: 50 mg triamterene loaded in 350 mg Gelucire, 150 mg silica and 50 mg alumina.

FIG. 5. Size distribution of drug-lipid droplets after dissolution of triamterene from active-release tablets in water.

### DETAILED DESCRIPTION OF THE INVENTION

This invention provides a composition in the form of a powder that facilitates dissolution and water dispersion of poorly soluble or insoluble compounds, and that, at the same time, are free flowing and compressible enough for pharmaceutical manufacturing processes such as tableting.

As used herein, free flowing is defined as that it can be easily processed with conventional pharmaceutical tableting, capsule-filling, or other formulation processes.

As used herein, compressible is defined as that it can be compressed to a tablet, dry granules or other formulations with appropriate mechanical strength and disintegration rates.

As used herein, poorly soluble is defined as the solubility of drug compounds due to which solubilization becomes the rate limit step for absorption of the drug. As a rule of thumb in pharmaceutical industry, such dissolution-related absorption problems can not be ruled out for a compound with an aqueous solubility less than 1% [12].

This invention provides the above composition which comprises a solid lipid or surfactant or a solid lipid mixture which may contain some liquid lipids, that dissolves water-insoluble or poorly soluble compounds and is able to be absorbed by a porous powder or a mixture of porous powders at melt state, and forms solutions, micelles, microemulsion or emulsion with the compounds in an aqueous medium.

As used in this invention, the porous powder or a mixture of porous powders is capable of absorbing melted lipids.

This invention also provides the above composition which comprises at least a compound that dissolves in the lipids and forms solutions, micelles, microemulsion or emulsion with the lipids in an aqueous medium.

This invention also provides the above composition wherein said the composition facilitates formation of solutions, micelles, microemulsions or emulsions of poorly soluble or water-insoluble compounds and the lipids after administration with no need of pre-emulsification of the compounds during formulation.

In an embodiment, the lipids are amphiphilic compounds. The lipids include but are not limited to Gelucire, vitamin E TPGS, Bay 10, fatty acids, phospholipids, and non-phospho-lipids.

This invention also provides the above composition wherein the porous powders are nontoxic solids possessing sufficient specific surface area and, pore structure.

In an embodiment, the surface area is bigger than 100 m²/g. In another embodiment, the surface area is at the range of 10 to 1000 m²/g

In a separate embodiment, the pore structure has a diameter less than 50 nm. In a further embodiment, the diameter is less than 10 nm. In a still further embodiment, the diameter is at a range of 2 to 1000 nm.

The pore structure includes but is not limited to alumina, silica, and cellulose derivatives

The compound includes but is not limited to cyclosporine, triamterene, acyclovir, doxorubicin, labetalol, doxepin, methyldopa and pentoxifill. As can easily appreciated by an ordinary skilled artisan in this field, this disclosure may be used with various compounds including active pharmaceutical compounds which are currently known and those which are to be developed.

This invention also provides a pharmaceutical composition comprising the above composition and a pharmaceutically acceptable carrier.

To produce the above composition, one can follow the below method:

a) Dissolving a compound in melted lipid or lipid mixtures;

b) Impregnating the said porous powders with the drug-lipid melt; and

c) Solidifying the drug-lipid melt absorbed in the porous powders by cooling, thereby producing the composition.

The above method may further comprise granulation, capsule filling, tableting, coating and paste making of the produced composition.

This invention also provides a composition produced by the above method and a pharmaceutical composition which comprises the produced composition.

The composition may be formulated in powders, capsules, granules, coated granules, tablets, or coated tablets.

The excipients of the formulated composition include but are not limited to binders, diluents, disintegrants, coating material, and lubricants.

The pharmaceutical compositions of the present invention comprise lipid molecules or mixed lipid molecules as carrier or carriers of poorly soluble drug substances. The lipid carrier system(s) dissolve(s) poorly soluble drugs at melt state due to their amphiphilic properties, and have the ability to form micelles, emulsions or microemulsions with the drugs upon hydration. Any nontoxic lipid which are solid at room temperature, preferably are solid up to 40° C., can be used as the lipid carrier systems. In the following examples, Gelucire 44/14 and Vitamin E TPGS were examined. Due to the amphiphilic nature, the lipid carrier systems absorb water to swell upon hydration, and dissolve, disperse (into water) or form micelles in water.

The present compositions also comprise porous solid materials or mixed materials which possess large specific surface area (from tens to hundreds m²/g), sufficient mechanic strength, and are pharmaceutically acceptable (no toxicity to humans) The average pore size of the porous materials with such large specific surface area is usually small (tens of nanometers). The porous materials also possess sufficient capacity (usually more than their own mass) to absorb the solution of drugs dissolved in melt lipid carriers (called drug-lipid melt hereafter). The porous materials are hydrophilic so that water can penetrate into their pore structure easily. Finally, the porous materials have good flow properties and compressibility after absorbing the drug-lipid melt(s), followed by cooling. As absorbents, a mixture of porous silicon dioxide and alumina are examined in the present invention for good balance between absorption capacity and mechanical strength. However, any pharmaceutical powders with sufficient surface area and pore volume are suitable for the present compositions.

Preparation of the compositions in the present invention involved following steps: 1) heat selected lipid materials or lipid mixture until the lipid materials completely melt; 2) dissolve poorly soluble drugs in the melted lipid sample; 3) add (impregnate) selected porous powders or mixed porous powders in the drug-lipid melt at a temperature higher than 40° C. until all the liquid be absorbed by the porous powder; 4) cool the sample to room temperature. The drug loaded powder can be further compressed with disintegrents to tablets or filled in capsules.

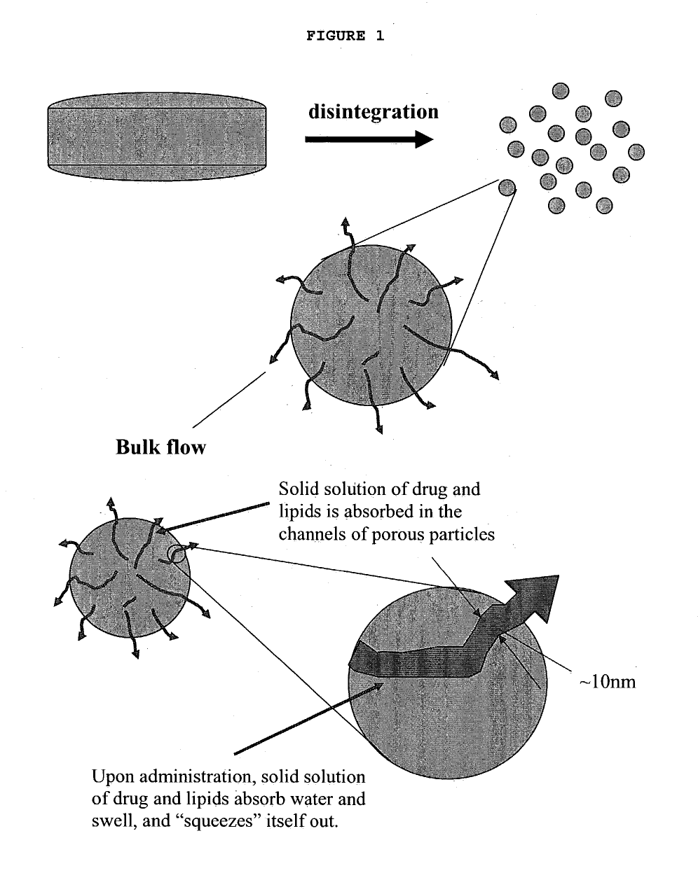

Although the pore structures of the porous powders are filled with the solidified drug-lipid melt, release of the poorly soluble drugs from the solid compositions is rapid. Moreover, our preliminary experiment showed that the time period required to achieve maximum release was independent of solubility of drugs loaded. These facts suggest a dissolution mechanism other than diffusion. It is hypothesized that hydration and swelling of the lipid carriers have played an important role in drug dissolution (See FIG. 1). Upon hydration, the lipid carriers swell in the nanometer-sized pores and squeezed themselves out of the pores.

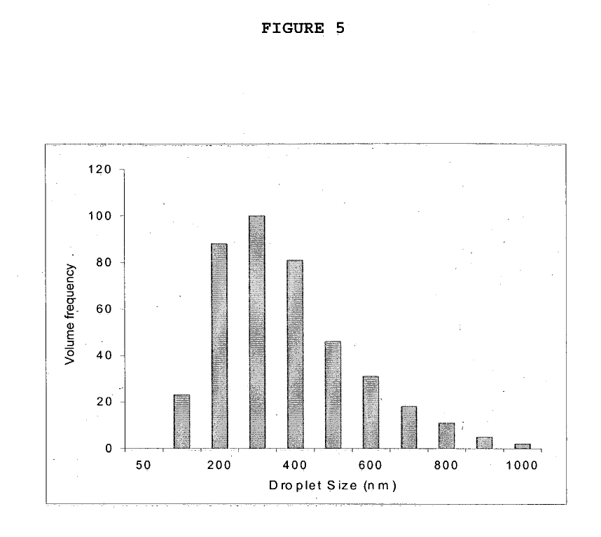

As shown by a laser scattering measurement of the dissolution medium (FIG. 5), under “insoluble” the condition (pH=7), the tramterene-lipid melt that was released from the present compositions formed droplets of a few hundred nanometers in diameter, typical sizes of the droplets in microemulsion. Hydrophobic drugs that form microemulsions are often regarded as apparently soluble and believed suitable for oral absorption [13]. With the present compositions, a self-emulsification process can easily be achieved without pre-forming a microemulsion prior to impregnation into the porous powders.

The compositions in the present invention possess a number of advantages over previously reported lipid-based solid dosage forms such as “microemulsion as solid dosage form” [10] , “powder solution” [7], lyophilized microemulsion [11], and drug-lipid hard gel capsules [9].

Compared with as “microemulsion as solid dosage form” [10] the present compositions can be prepared with no need of forming a microemulsion prior to loading onto solid powders and no need of water evaporation after impregnation of the powders. Formation of a microemulsion is not feasible for many drugs and the use of organic solvents is often required. For a given dose, the total volume of the drug-lipid melts is much smaller than that of a microemulsion (since additional water is required to for the continuous phase, the majority volume of an emulsion). In the case of the present compositions, all the pore-volume of the porous materials is filled by drug and lipids only during impregnation. Therefore, for same-drug loadings, the overall size of a solid dosage form made of the compositions in the present invention is much smaller that that prepared through a pre-formed microemulsion. This nature makes the present compositions feasible for many poorly soluble drugs for which the dose is too large to use the “microemulsion as solid dosage form” [10]. In these compositions, the total mass of drugs and lipids to be absorbed is more than that of the absorbents and the porous powders.

Differing from the so-called “powder solutions” [7] which are liquid-in-solid systems, the compositions in the present invention are real solids. The liquid-in-solid dosage forms suffer from instability, poor flow property and erratic compressibility, thus are only feasible for low-dose drugs. These problems are not associated with the present compositions due to their solid nature.

The compositions in the present invention are also superior over the drug-lipid capsules, although same lipid carriers may be used. For a drug-lipid capsule, the drug dissolution is through surface erosion of the plug form by the drug and the lipid carriers, thus it is relatively slow. Moreover, the drugs dissolved in the lipid matrix migrate and form crystals [9]. For the present compositions, the drug-lipid melts are dispersed and isolated in each nanometer sized pores. In addition to fast dissolution (normally within 30 min), the drug molecules isolated in each pore are not sufficient to form crystals.

The lyophilized microemulsion [11] may be a solution for delivery of insoluble drugs through injection. For oral dosage form, lyophilization of a microemulsion is regarded as an unnecessary and costly manufacturing process.

The above-mentioned advantages of the novel compositions of the present invention are achieved without compromise to any function seen in lipid-assistant oral dosage forms. Any drug-lipid combination, as long as they form apparent solutions (microemulsion, solution, micelles) are suitable to be formulated with the present compositions. The ability of lipids or other surfactants to improve solubility, absorption and bioavailability of poorly drugs are fully preserved. Examples of drugs suitable for this system include, but are not limited to cyclosporine, triamterene, acyclovir, doxorubicin, labetalol, doxepin, methyldopa, and pentoxifill. This type of composition is also suitable for pharmaceutical compounds under development which have solubility problems.

Other pharmaceutical ingredients, such as binders, disintegrants, or coating materials, may be preferably used with the present dosage forms. These ingredients include but are not limited to microcrystalline cellulose, croscarmelose sodium, crospovidone, starch, methylcellulose A, sodium alginate, and cellulosephthalate.

The compositions of this invention can be formulated to various solid-dosage forms, such as tablets, coated tablets, hard capsules, and granules using conventional methods.

The invention will be better understood by reference to the Examples which follow, but those skilled in the art will readily appreciate that the specific experiments detailed are only illustrative, and are not meant to limit the invention as described herein, which is defined by the claims which follow thereafter.

### EXAMPLES

### Example 1

Granule Characteristics of Alumina-Cab-o-Sil Mixture Impregnated with Gelucire 44/14

The capacity of Alumina-Cab-O-Sil mixture in absorbing lipid melts and its effect on granule properties were examined systematically. Gelucire, 3 g, was placed in each beaker and melted at 80° C. using an oil bath. Then Alumina-Cab-O-Sil mixture with alumina to silica ratio at 1:1, 1:2, 1:3 and 1:4, and with total weight of 1.5 g, 1.6 g, 1.7 g, 1.8 g, 1.9 g and 2.0 g were added into each beaker with the melted Gelucire. After the melted lipid was absorbed into the oxide mixture, the samples were cooled to room temperature to solidify for three hours. The flow properties and compressibility of the solidified powders were examined using angle of repose and a tablet hardness tester to find the maximum capacity of the Alumina-Cab-o-Sil mixture in absorbing lipids. The results are summarized in Table 1.

TABLE 1
Physical Properties of Alumina-Silica Mixture
Impregnated with Gelucire

| Gelucire impregnation by Alumina | | Granule | | | |
|---|---|---|---|---|---|
| and Cab-O-Sil mixtures | | Characteristics | | | |
| Amount | Amount | Ratios of Alumina | | | |
| of | of Alumina | Cab-O- | | | |
| Gelucire | Cab-O-Sil | Sil Mixture | | | |
| (g) | Mixture (g) | 1:1 | 1:2 | 1:3 | 1:4 |
| 3 | 1.5 | x | x | x | x |
| 3 | 1.6 | x | x | x | ✓ |
| 3 | 1.7 | x | x | ✓ | ✓ |
| 3 | 1.8 | x | x | ✓ | xx |
| 3 | 1.9 | x | x | xx | xx |
| 3 | 2.0 | x | ✓ | xx | xx |

x → Unsuitable because of grittiness
✓ → Suitable for subsequent process
xx → Unsuitable because of excessive fine powder

Alumina-Cab-O-Sil mixtures of 1:2 alumina to silica ratio and 2.0 g total weight, 1:3 ratio and 1.7 to 1.8 g, and 1:4 ratio and 1.6 to 1.7 g were found to provide the optimal results. The hardness of compressed tablets varied with the alumina to silica ratio as 1:2:>20 kp; 1:3:˜10 kp; and 1:4:<4 kp. Oxide mixture with alumina to silica ratio of 1:3 was used for successive experiments.

### Example 2

Solubility and Water Dispersity of Triamterene-Gelucire 44/14 (Mixed Lipids)

To examine the compositions described above, triamterene was used as a model drug. Tiamterene (MW=253 and pKa=6.2) slightly dissolve at pH 1 (321 ug/ml) buffer and practically insoluble at pH 7 (45 ug/ml), and shows light a maximum absorbance at 357 nm. The compounds are easy to detect and possess a wide solubility range as a function of pH. In experiment, 30 mg triamterene was dissolved in various amounts of melted Gelucire to make the drug concentration to be 5%, 7.5%, 10% 12.5% 15% and 20%, respectively. Then the drug-lipid melts were dissolved in 900 ml of water. Up to 10% of triamterene in Gelucire, the drug was dispersed in water without precipitates. At higher drug contents (i.e. less lipids), precipitates were observed under a microscope. Drug concentration (in Gelucire) of 10% was selected-for later experiments.

### Example 3

Dissolution Profile of Triamterene from Active-Release Tablet Compared with other Dosage Forms in 0.1 M HCl.

Since triamterene slightly dissolved at low pH medium, 0.1 M HCl was used as the medium to compare dissolution kinetics between tablet form of the compositions of the present invention (called active-release tablets hereafter) and other formulations as references. Active-release tablets each of which contained 30 mg triamterene, 350 mg Gelucire 44/14, 150 mg silica (Cab-O-Sil, 200 m²/g), 50 mg alumina, 250 mg Emcocel LP2000, 60 mg calcium phosphate dehydrate and 60 mg crosscarmelose sodium (regarded as “super disintegrant”) were prepared for dissolution test. Triamterene was first dissolved in melted Gelucire then absorbed in the mixture of alumina and silica prior to tableting. For comparison, a “reference tablet” containing the same ingredients as an active-release tablet except Gelucire and the alumina-silica mixture was prepared by direct compressing. In addition, capsules containing 30 mg triamterene only (called drug-only capsule hereafter), and containing 30 mg and 350 Gelucire (called drug-lipid plug capsule or plug capsule hereafter) were prepared.

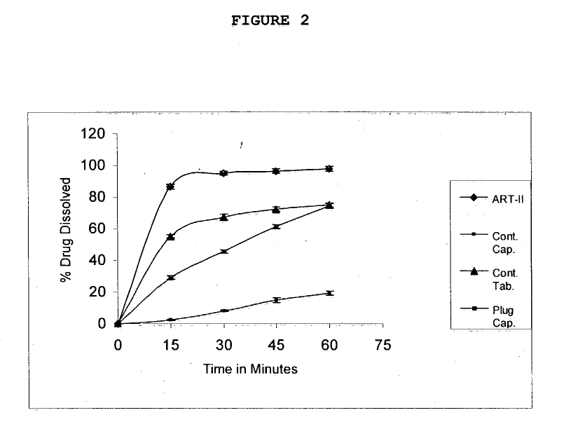

For the dissolution study, the four formulations were suspended in 900 ml 0.1 M HCl at 37° C. with stirring gat 100 rpm. The aliquots collected as a function of time were filtrated through a 0.45 um Millipore filter and diluted with 0.1 M HCl prior to subjecting to a photometer at 357 nm. The result is shown in FIG. 2. For Active-release tablet, more than 80% triamterene was released with 15 min of dissolution, and release completed at 30 min sampling. The initial dissolution rate (average of 15 min) was 5.7%/min. For the reference tablet, 55% of triamterene loading was released in the first 15 min, followed by a reduced release rate that lead to 75% release cumulatively for 60 min. The initial release rate was 3.7%/min. The plug capsule showed a gradual release profile with initial release rate of 1.9% and cumulatively 74% was released for 60 min. The drug-only capsule without any excipients showed the least dissolution rate (initial rate: 0.2%, 60 min cumulative release: 19%). The active-release tablet showed significantly increased dissolution rate.

### Example 4

Water Dispersion Profile of Triamteren from Active-Release Tablet as Compared with other Dosage Forms in Water.

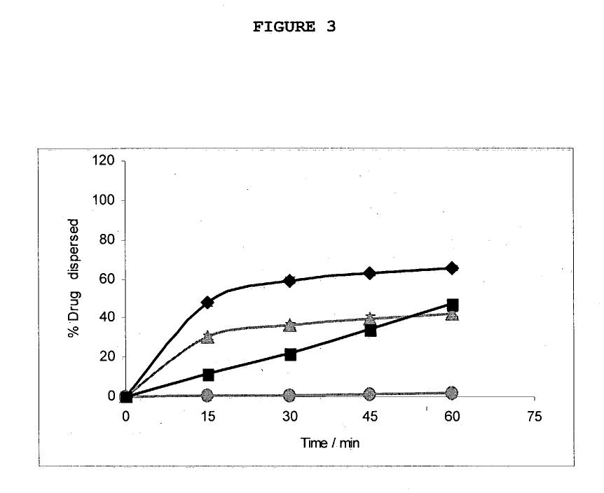

To examine how the active-release tablet affects dissolution of insoluble drugs, dissolution of triamterene was carried out in 900 ml water (pH=7) and compared with reference formulations. All the formulations were prepared with the same content and procedure as in Example 3, respectively. The released drug amount was measured by absorbance of the supernatant at 357 nm as function of time. The result is shown in FIG. 3. Similar patterns of drug release were observed for the four formulations as in Example 3, respectively. However, the cumulative release for 60 min was lower in water as compared with that in 0.1 M HCl for all the samples. In addition, the release medium was cloudy when water was used as the medium. The aliquots collected as a function of time were centrifuged at 1000 rpm in an Eppendorf tube for 1 min to remove the alumina-silica particles. The supernatant was diluted with 0.1 M HCl and analyzed photometrically at 357 nm. The result is shown in FIG. 3. For active-release tablet, the initial dissolution rate in water was 3.2%/min, and cumulative release for 60 min was 66%. Following, active-release tablet were the reference tablet, plug capsule and drug-only capsule which showed initial release rate of 2%, 0.8% and 0.03%, and 60 min-cumulative of 42%, 47% and 2%, respectively. Clearly, the active-release tablet (made of the compositions of the present invention) significantly facilitated dissolution of insoluble drugs (triamterene is insoluble in medium of pH=7).

### Example 5

Drug Loadings and Dissolution Profile

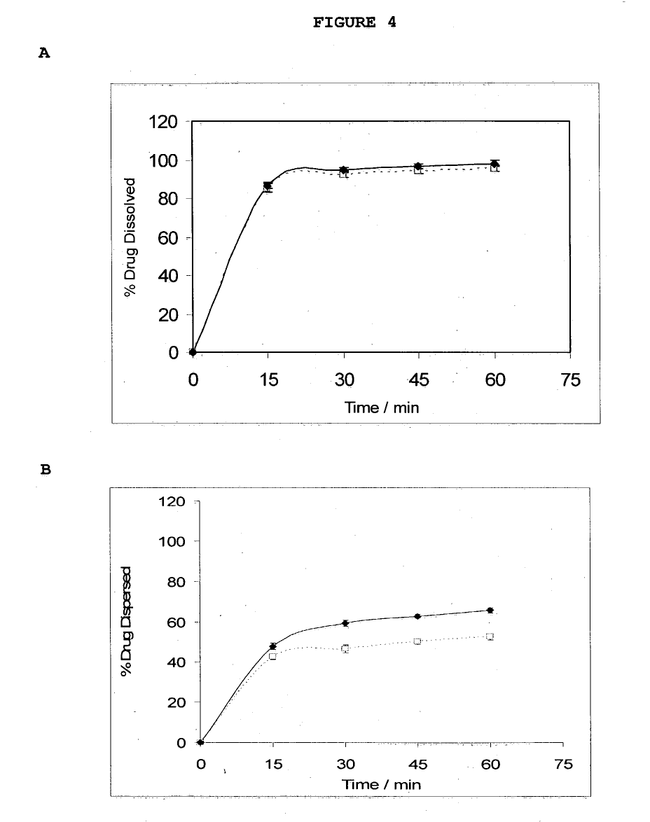

To elucidate why only 66% triamterene released from the active-release tablet in 60 min, the loading the triamterene was increased from 30 mg to 50 mg with other ingredients unchanged (See Example 3). Dissolution study of the tablets of different triamterene loadings was carried out at pH=1 and pH=7, respectively. In the medium of pH=1, there was no difference regarding drug release profile between the tablets of different drug loadings (See FIG. 4A). However, at pH=7, there were significant differences in the release profiles between the two tablets of different triamterene loadings (See FIG. 4B). Increase in drug loading resulted in a decrease in dissolution rate and apparent solubility of triamterene. This decrease in percentage release is probably due to increased drug-to-lipid ratio.

### Example 6

Size Distribution of Drug-Lipid Droplets Released from Active-Release Tablets.

The morphologies of triamterene after dissolution in 0.1 M HCl and in water were characterized using a sub-micron particle sizer. The aliquots collected at the end of dissolution were centrifuged under the same conditions as in Example 4 prior to measurement. For dissolution medium of 0.1 M HCl, no particles were detected. For the sample of dissolution in water, however, droplets with mean diameter of 277 nm were observed (See FIG. 5). This is probably because triamterene is soluble at pH=1 but insoluble at pH=7. This result also indicates that the composition of the present invention could apparently dissolve insoluble drugs in the form of microemulsion.

### Example 7

Dissolution Test of Cyclosporin A from Active-Release Tablets

Cyclosporin A was selected to further examine the applicability of the composition of this invention for insoluble drugs. Cyclosporin A, 25 mg, was dissolved in a mixture of Gelucira (125 mg) and Vitamin E TPS (125 mg) at 80° C., followed by impregnation of dried mixture of Carb-O-Sil (100 mg) and alumina (30 mg) into the drug-lipid melt. A dry-powder with good flow property was obtained after the drug-lipid melt was absorbed into the porous oxides and cooled down to room temperature. The impregnated powder was suspended in 900 ml PBS buffer at 37° C. with stirring at 100 rpm. For comparison, a drug-lipid plug with the same masses of cyclosporine A, Gelucire and vitamin E TPGS was subjected to dissolution under the same condition. The aliquots were collected at programmed time intervals, followed by centrifugation at 1000 rpm in an Eppendorf tube for 1 min. The obtained supernatants were analyzed using a Shimazu HPLC with reversed phase column and a mobile phase consisting of 10% methanol, 40% acetonitrile and 50% water. As the result, the drug-lipid plug reached 60% release cumulatively for 60 min of dissolution, while that formulated with the present composition reached 80% release for the same time period. Again, the composition of the present invention facilitated apparent dissolution of an insoluble drug significantly. The droplet sizes of cyclosporine A after dissolution was ranged at 50-220 nm as measured in the same procedure in Example 6, indicating formation of a microemulsion.

### REFERENCES

[1] S. C. Mehta, “Issues and approaches for improving the solubility and bioavailability of poorly soluble compounds”, Bulletin Tech., Gattefose, No. 91, 65-71 (1998).

[2] D. A. Wyatt, “Taking poorly water soluble compounds through discovery”, Bulletin Tech Gattefose, No. 92, 31-39 (1999).

[3] E. Nelson, J. Pharm. Sci., 47, 297 (1958).

[4] E. Nelson, S. Long, J. G. Wagner, J. Pharm. Sci., 53, 1224 (1964).

[5] S. Niazi, J. Pharm. Sci., 65, 302 (1976).

[6] A. T. M. Serajuddin, “Physicochemical basis of increased bioavailability of a poorly water-soluble drug during following oral administration of organic solutions”; J. Pharm. Sci., 77(4), 325-329 (1988).

[7] A. Sheth, I. Jarowski, “Use of powder solution to improve the dissolution rate of polythiazide tablets”; Drug Development and Industrial Pharmacy, 16 (5), 965-977 (1990).

[8] S. K. Dordunoo, “Preformulation studies on solid dispersions containing triamterene or temazepam in polyethylene glycols or Celucire 44/14 for liquid filling hard gelatin capsules”; Drug Development and Industrial Pharmacy, 17(12), 1685-1713 (1991).

[9] A. T. M. Serajuddin, “Solid dispersion of poorly water soluble drugs: early promises, subsequent problems, and recent breakthroughs”; J. Pharm. Sci., 88(10), 1058-1066 (1999).

[10] Pather, et al., “Microemulsion as solid dosage forms for oral administration”; U.S. Pat. No. 6,280,770 (2001).

[11] B. Lundberg, “A submicron lipid emulsion coated amphipathic polyethylene glycol for parental administration of paclitael”, J. Pharm. Pharmacol., 49, 16-21 (1997).

[12] D. A. Wadke, A. T. Serajuddin, H. Jacobson, “Preformulation testing”; in Pharmaceutical Dosage Forms: Tablets, pg 12-13, Ed. H. A. Lieberman, et al., Marcel Dekker, 1989, New York

[13] P. P. Constanides, “Lipid microemulsions for improving drug dissolution and oral absorption: physical and biopharmaceutical aspects”; Pharm. Res., 12(11), 1561-1572 (1995).

What it claimed is:

1. A composition in the form of a free flowing, compressible powder that facilitates dissolution and water dispersion of poorly soluble or insoluble compounds.

2. The composition of claim 1 comprising a solid lipid or a solid lipid mixture that dissolves water-insoluble or poorly soluble compounds and is able to be absorbed by a porous powder or a mixture of porous powders at melt state, and forms solutions, micelles, microemulsion or emulsion with the compounds in an aqueous medium.

3. The composition of claim 1 comprising a porous powder or a mixture of porous powders that absorb melted lipids.

4. The composition of claim 1 comprising, at least, a compound that dissolves in the lipids and forms solutions, micelles, microemulsion or emulsion with the lipids in an aqueous medium.

5. The composition of claim 1 wherein said the composition facilitates formation of solutions, micelles, microemulsions or emulsions of poorly soluble or water-insoluble compounds and the lipids after administration with no need of pre-emulsification of the compounds during formulation.

6. The composition of claim 2 wherein the lipids are amphiphilic compounds.

7. The composition of claim 6, wherein the lipid is Gelucire, vitamin E TPGS, Bay 10, fatty acids, phospholipids, or non-phospholipids.

8. The composition of claim 3, wherein the porous powders are nontoxic solids possessing sufficient specific surface area and,pore structure.

9. The composition of claim 8, wherein the surface area is bigger than 100 m²/g.

10. The composition of claim 8, wherein the pore structure has a diameter less than 50 nm).

11. The composition of claim 10, wherein the pore structure is alumina, silica or cellulose derivatives

12. The composition of claim 4, wherein the compound is cyclosporine, triamterene, acyclovir, doxorubicin, labetalol, doxepin, methyldopa or pentoxifill.

13. A pharmaceutical composition comprising the composition of claim 1-12 and a pharmaceutically acceptable carrier.

14. A method for producing the composition of claim 1, comprising steps of:
d) Dissolving the said compound in melted lipid or lipid mixtures;
e) Impregnating the said porous powders with the drug-lipid melt; and
f) Solidifying the drug-lipid melt absorbed in the porous powders by cooling, thereby producing the composition.

15. The method of claim 14, further comprising granulation, capsule filling, tableting, coating and paste making of the produced composition.

16. The composition produced by the method of claim 14.

17. A pharmaceutical composition which comprises the composition of claim 16.

18. The composition of claim 16, formulated in powders, capsules, granules, coated granules, tablets or coated tablets.

19. The formulated composition of claim 18, comprising the excipients selected from the group containing binders, diluents, disintegrants, coating material, and lubricants.
